(19) United States
(12) Patent Application Publication
(10) Pub. No.: US 2020/0064349 A1
(43) Pub. Date: Feb. 27, 2020

(54) PROSTATE CANCER DIAGNOSTIC BIOMARKER COMPOSITION INCLUDING KYNURENINE PATHWAY'S METABOLITES

(71) Applicants: Korea University Research and Business Foundation, Sejong Campus, Sejong (KR); Industry-Academic Cooperation Foundation, Yonsei University, Seoul (KR)

(72) Inventors: Youngja PARK HWANG, Sejong-si (KR); Adnan KHAN, Sejong-si (KR); Jinhyuk NA, Sejong-si (KR); Sun ha JEE, Seoul (KR)

(73) Assignees: Korea University Research and Business Foundation, Sejong Campus, Sejong (KR); Industry-Academic Cooperation Foundation, Yonsei University, Seoul (KR)

(21) Appl. No.: 16/549,029

(22) Filed: Aug. 23, 2019

(30) Foreign Application Priority Data
Aug. 24, 2018 (KR) 10-2018-0099349

(51) Int. Cl. G01N 33/574

(52) U.S. Cl. CPC G01N 33/57434; G01N 33/57484

(57) ABSTRACT

Disclosed is a prostate cancer diagnostic biomarker composition including, as an active ingredient, at least one kynurenine pathway metabolite selected from kynurenine and anthranilate. Also disclosed are a prostate cancer diagnostic composition including an agent capable of measuring the level of the biomarker and a prostate cancer diagnostic kit including the prostate cancer diagnostic composition. The use of the prostate cancer diagnostic biomarker composition can significantly improve the accuracy of prostate cancer diagnosis as an alternative or supplement to the use of PSA levels for prostate cancer diagnosis according to the prior art, which has difficulty in ensuring accurate diagnosis. Therefore, the biomarker composition can be widely used in a variety of industrial applications, including medical applications.

### CROSS-REFERENCE TO RELATED APPLICATIONS

This application claims the benefit under 35 USC 119(a) of Korean Patent Application No. 10-2018-0099349 filed on Aug. 24, 2018 in the Korean Intellectual Property Office, the entire disclosure of which is incorporated herein by reference for all purposes.

### BACKGROUND OF THE INVENTION

### 1. Field of the Invention

The present invention relates to a prostate cancer diagnostic biomarker composition including at least one kynurenine pathway metabolite. More specifically, the present invention relates to a prostate cancer diagnostic biomarker composition including, as an active ingredient, at least one kynurenine pathway metabolite selected from kynurenine and anthranilate, a prostate cancer diagnostic composition including an agent capable of measuring the level of the biomarker, a prostate cancer diagnostic kit including the prostate cancer diagnostic composition, and a method for providing information on prostate cancer diagnosis including (a) measuring the level of a biomarker including at least one kynurenine pathway metabolite selected from kynurenine and anthranilate in a biological sample from an individual and (b) comparing the measured biomarker level with that in a biological sample from a control.

### 2. Description of the Related Art

Prostate cancer (PCa) has become one of the most common causes of cancer-related death in men in the USA. Prostate, respiratory, and colorectal cancers combined were expected to have caused 46% of all cancer-related deaths in 2016. Particularly, PCa was reported to be associated with factors related to economic development and indeed has become a more pressing problem in the populations of many developing Asian countries.

PCa initially develops in prostatic cells and can spread to nearby tissues, as well as other organs. The most frequent metastasis sites are bone, lung, liver, pleura, and adrenal gland. Several risk factors that may increase the chance of acquiring PCa include genetics and environmental factors. Existing PCa screening methods use a digital rectal examination in combination with the determination of serum prostate-specific antigen (PSA) levels, followed by a diagnostic biopsy. This method reveals most malignancies, but the efficacy of routine PSA level screening has recently been questioned. Approximately only 25% of men with an elevated PSA level (>4.0 ng/mL) are diagnosed with PCa after biopsy, with false-negatives being a common occurrence. Moreover, biopsies do not always identify cancer because of tumor heterogeneity, imposing the need for multiple biopsies that are potentially dangerous for patients. Additionally, the final recommendation statement of the U.S. Preventive Services Task Force (USPSTF) was against PSA-based screening for prostate cancer. As many healthy men in PSA screened population experienced the harms of biopsies and treatment than the benefit. In addition, the Gleason score, a worldwide grading scheme based on the histological pattern of the display of carcinoma cells in prostate biopsies, has been shown to be higher in Korean and Asian-American subjects than that in other ethnic groups, despite equal access to health care. Therefore, the discovery of a new biomarker capable of enhancing the diagnosis of prostate cancer with the supplement of PSA level determination is believed to improve the current strategies available to manage prostate cancer.

Biomarkers are molecules present in biological fluids, and their detection can provide information about a disease that may not be obtained through analysis of standard clinical parameters. In addition to proteins and metabolites, RNA transcripts, DNA, or epigenetic modifications of DNA can be used as biomarkers. Metabolomics has been used for biomarker discovery in the life, plant/food, and environmental sciences. Recently developed configurations such as quadrupole time-of-flight (Q-TOF) tandem mass spectrometry in combination with liquid chromatography have improved metabolite screening by enhancing both mass resolution and mass accuracy.

Previous studies have shown PCa progression and recurrence of PCa even in the presence of undetectable or low serum PSA level. However, the predictive value of PSA in the range of 0.0 to 4.0 and the exact mechanism for the low specificity of PSA levels in cancer patients is still unknown. In addition, no high-resolution metabolomics (HRM) study, until date, has investigated the impact of low and/or high PSA levels in PCa pathogenesis on metabolic alterations.

Thus, the present inventors have earnestly and intensively conducted research to solve the problems of the prior art. As a result, the present inventors have confirmed that the discovery of specific kynurenine pathway metabolites enables efficient detection of prostate cancer and have found that new biomarkers including kynurenine, anthranilate, and their peripheral metabolites can be used as alternative or auxiliary diagnostic indices for uncertain diagnosis by prostate-specific antigen (PSA). The present invention has been accomplished based on this finding.

### SUMMARY OF THE INVENTION

One object of the present invention is to provide a prostate cancer diagnostic biomarker composition including at least one kynurenine pathway metabolite as an active ingredient wherein the kynurenine pathway metabolite is kynurenine and/or anthranilate.

A further object of the present invention is to provide a prostate cancer diagnostic composition including an agent capable of measuring the level of the biomarker.

Another object of the present invention is to provide a prostate cancer diagnostic kit including the prostate cancer diagnostic composition.

Still another object of the present invention is to provide a method for providing information on prostate cancer diagnosis including (a) measuring the level of a biomarker including at least one kynurenine pathway metabolite selected from kynurenine and anthranilate in a biological sample from an individual and (b) comparing the measured biomarker level with that in a biological sample from a control.

One aspect of the present invention provides a prostate cancer diagnostic biomarker composition including at least one kynurenine pathway metabolite as an active ingredient wherein the kynurenine pathway metabolite is kynurenine and/or anthranilate.

A further aspect of the present invention provides a prostate cancer diagnostic composition including an agent capable of measuring the level of the biomarker.

Another aspect of the present invention provides a prostate cancer diagnostic kit including the prostate cancer diagnostic composition.

Yet another aspect of the present invention provides a method for providing information on prostate cancer diagnosis including (a) measuring the level of a biomarker including at least one kynurenine pathway metabolite selected from kynurenine and anthranilate in a biological sample from an individual and (b) comparing the measured biomarker level with that in a biological sample from a control.

The use of the prostate cancer diagnostic biomarker composition can significantly improve the accuracy of prostate cancer diagnosis as an alternative or supplement to the use of PSA levels for prostate cancer diagnosis according to the prior art, which has difficulty in ensuring accurate diagnosis. Therefore, the biomarker composition of the present invention can be widely used in a variety of industrial applications, including medical applications.

### BRIEF DESCRIPTION OF THE DRAWINGS

These and/or other aspects and advantages of the invention will become apparent and more readily appreciated from the following description of the embodiments, taken in conjunction with the accompanying drawings of which:

FIG. 1 shows multivariate statistical analyses to distinguish the metabolic phenotypes between the control and PCa patients. Separation and classification of the metabolic profiles between control and PCa patients with PSA <4 or PSA >4 by (FIG. 1A) unsupervised Principal Component Analysis (PCA) and (FIG. 1B) supervised Partial Least Squares Discriminant Analysis (PLS-DA), and (FIG. 1C) heat map produced by hierarchical clustering of the top differential metabolite features. Red dots or panels represent control (n=96), green represent PCa patients with PSA level <4 (n=25), and blue represent PCa patients with PSA level >4 (n=25);

FIG. 2 shows multivariate statistical analyses to discriminate the metabolic phenotypes between PCa patients with differential PSA levels. Separation and classification of the metabolic profiles between PCa patients with PSA level <4 and those with PSA level >4 by (FIG. 2A) PCA and (FIG. 2B) PLS-DA, and (FIG. 2C) heat map produced by hierarchical clustering of the top differential metabolite features. Green dots or panels represent PCa patients with PSA level <4 (n=25), and blue represent PCa patients with PSA level >4 (n=25);

FIG. 3 shows multivariate statistical analyses to discriminate the metabolic phenotypes between control and PCa patients with PSA level <4. Separation and classification of the metabolic profiles between control and PCa patients with PSA <4 by (FIG. 3A) unsupervised Principal Component Analysis (PCA) and (FIG. 3B) supervised Partial Least Squares discriminant analysis (PLS-DA), and (FIG. 3C) heat map produced by hierarchical clustering of the top differential features. Red dots or panels represents control (n=96), green represents PCa patients with PSA level <4 (n=25);

FIG. 4 shows multivariate statistical analyses to discriminate the metabolic phenotypes between control and PCa patients with PSA level >4. Separation and classification of the metabolic profiles between control and PCa patients with PSA>4 by (FIG. 4A) unsupervised Principal Component Analysis (PCA) and (FIG. 4B) supervised Partial Least Squares discriminant analysis (PLS-DA), and (FIG. 4C) heat map produced by hierarchical clustering of the top differential features. Red dots or panels represents control (n=96), and blue represents PCa patients with PSA level >4 (n=25);

FIG. 5 shows top 10 affected pathways between control vs PCa patients with PSA <4 or PSA >4 in KEGG analysis. Y-axis label represents pathway's names and X-axis label represents the number of hits in the respective pathways;

FIG. 6 shows Manhattan plots and pathway identification using Mummichog. (FIG. 6A) Visualized significant metabolites with Manhattan plot according to m/z (left) and retention time (right). 2,362 of 5,312 features were found to be significant (p<0.05). The green dots on the dashed line represent significant features. (FIG. 6B) Pathway analysis based on the significant 2,362 metabolites. Tryptophan metabolism was detected as the most significant pathway, with −log (p) value of 0.0005;

FIG. 7 shows pathway overview and relative concentrations of significant tryptophan metabolism metabolites in healthy and cancer patients. Relative concentrations of significant metabolites involved in tryptophan metabolism along the kynurenine pathway, namely, L-tryptophan (m/z: 227.07 [M+H]⁺), kynurenine (m/z: 209.09 [M+H]⁺), anthranilate (m/z: 138.05 [M+H]⁺), isophenoxazine (m/z: 235.04 [M+Na]⁺), glutaryl-CoA (m/z: 864.14 [M+H−H₂O]⁺), (S)-3-hydroxybutanoyl-CoA (m/z: 871.17 [M+NH₄]⁺), acetoacetyl-CoA (m/z: 852.13 [M+H]⁺), and acetyl-CoA (m/z: 832.12 [M+Na]⁺), and tryptophan metabolism metabolites along the alternate pathway, namely, indoxyl (m/z: 156.04 [M+Na]⁺), indolelactate (m/z: 188.06 [M+H−H₂O]⁺), and indole-3-ethanol (m/z: 144.08 [M+H−H₂O]⁺). The bar graphs represent the metabolites whose levels show significant differences in the following 3 groups: control subjects, PCa patients with PSA level <4, and PCa patients with PSA level >4. ***p<0.001; **p<0.01; *p<0.05; ns not significant (p>0.05) Student's t test;

FIG. 8 shows identification and validation of tryptophan by LC-ESI/MS/MS. The tryptophan fragmentation was observed in the standard and serum samples of PCa patients in the positive mode on a UHPLC-Q-TOF-LC/MS with collision energy of 5, 10, 15, and 20 V. (FIG. 8A) EIC scan mode showing tryptophan peaks of standard and serum samples of PCa patients. The intensity of peaks was increased at 3 min. (FIG. 8B) revealed that product-ion analysis of tryptophan in standard reference at 15 V, serum sample obtained from PCa patients with PSA level <4 at 15 V, and serum sample obtained from PCa patients with PSA level >4 at 15 V produced the same patterns of tryptophan (ESI, electron spray ionization; CID, collision-induced dissociation; EIC, extracted ion chromatogram; rt, retention time; frag, fragmentor voltage);

FIG. 9 shows identification and validation of kynurenine by LC-ESI/MS/MS. The kynurenine fragmentation was observed in the standard and serum samples of PCa patients in the positive mode based on UHPLC-Q-TOF-LC/MS with collision energy of 5, 10, 15, and 20 V. (FIG. 9A) EIC scan mode showing kynurenine peaks of standard and serum samples of PCa patients. The intensity of peaks was increased at 1.6 min. (FIG. 9B) revealed that product-ion analysis of standard reference at 10 V, serum sample obtained from PCa patients with PSA level <4 at 10 V, and serum sample obtained from PCa patients with PSA level >4 at 10 V produced the same patterns of kynurenine (ESI, electron spray ionization; CID, collision-induced dissociation; EIC, extracted ion chromatogram; rt, retention time; frag, fragmentor voltage);

FIG. 10 shows identification and validation of anthranilate by LC-ESI/MS/MS. The anthranilate fragmentation was observed in the standard and serum samples of PCa patients in the positive mode based on UHPLC-Q-TOF-LC/MS with collision energy of 5, 10, 15, and 20 V. (FIG. 10A) EIC scan mode showing anthranilate peaks of standard and serum samples of PCa patients. The intensity of peaks was increased at 3.8 min. (FIG. 10B) revealed that product-ion analysis of standard reference at 15 V, serum sample obtained from PCa patients with PSA level <4 at 15 V, and serum sample obtained from PCa patients with PSA level >4 at 15 V produced the same patterns of anthranilate (ESI, electron spray ionization; CID, collision-induced dissociation; EIC, extracted ion chromatogram; rt, retention time; frag, fragmentor voltage); and

FIG. 11 shows quantified concentrations of tryptophan, kynurenine, and anthranilate in serum samples by LC-ESI-MS/MS, specifically concentrations of tryptophan (FIG. 11A), kynurenine (FIG. 11B) and anthranilate (FIG. 11C) in serum samples from control or PCa patients, in reference to the calibration curve of each standard compound. Concentrations of each compound were calculated by reference to the peak areas of the external standards within the range of LOD and LOQ. **p<0.01; *p<0.05; ns not significant (p>0.05) Student's t test.

FIG. 12 shows the quantified concentrations of tryptophan (FIG. 12A), kynurenine (FIG. 12B) and anthranilate (FIG. 12C) in serum samples from training set's control (n=100) or PCa patients (n=50) with PSA level <4 (n=37) and with PSA level >4 (n=13). Concentrations of tryptophan, kynurenine and anthranilate in serum were calculated in reference to the calibration curve of each standard compound. Concentrations of each compound were calculated by reference to the peak areas of the external standards within the range of LOD and LOQ. **p<0.01; *p<0.05; ^{ns}not significant (p>0.05)-student's t test.

### DETAILED DESCRIPTION OF THE INVENTION

Unless otherwise defined, all technical and scientific terms used herein have the same meaning as commonly understood by one of ordinary skill in the art to which this invention belongs. In general, the nomenclature used herein is well known and commonly employed in the art.

Using LC-MS based HRM, the present inventors elucidated differential metabolic phenomena in PCa patients who were with elevated or reduced serum PSA levels in the Examples section that follows. Specifically, it was found that the expression of tryptophan, indoxyl, kynurenine, anthranilate, isophenoxazine, glutaryl-CoA, (S)-3-hydroxybutanoyl-CoA, acetoacetyl-CoA, and acetyl-CoA was upregulated in correlation with the PSA level in prostate cancer patients, but the expression of indolelactate and indole-3-ethanol were downregulated in the prostate cancer patients.

In one aspect, the present invention is directed to a prostate cancer diagnostic biomarker composition including at least one kynurenine pathway metabolite as an active ingredient wherein the kynurenine pathway metabolite is kynurenine and/or anthranilate.

The term “biomarker” as used herein refers to a substance that can differentially diagnose prostate cancer from other non-prostate cancer conditions and is intended to include organic biomolecules such as polypeptides, nucleic acids, lipids, glycolipids, glycoproteins, sugars, and proteins whose levels are elevated or reduced in samples from individuals with prostate cancer compared to in samples from individuals without prostate cancer. In the present invention, the prostate cancer diagnostic biomarker may be a kynurenine pathway metabolite. The biomarker is preferably selected from the group consisting of kynurenine, anthranilate, tryptophan, indoxyl, indole-3-ethanol, indolelactate, isophenoxazine, glutaryl-CoA, (S)-3-hydroxybutanoyl-CoA, acetoacetyl-CoA, and acetyl-CoA. More preferably, the biomarker is kynurenine or anthranilate.

The term “diagnosis” as used herein refers to the identification of the presence or properties of pathological states. In the present invention, the diagnosis may mean the identification of development, progression or relapse of prostate cancer.

In a further aspect, the present invention is directed to a method for providing information on prostate cancer diagnosis including (a) measuring the level of a biomarker including at least one kynurenine pathway metabolite selected from kynurenine and anthranilate in a biological sample from an individual and (b) comparing the measured biomarker level with that in a biological sample from a control.

The term “individual” as used herein refers to a subject or patient and may be a mammal or non-mammal.

The term “biological sample” as used herein is intended to include, but is not limited to, tissue, cell, whole blood, plasma, serum, blood, saliva, lymph, and urine.

The terms “level” and “value” as used herein are used interchangeably to refer to a measurement that is made using any analytical method for detecting the biomarker in the biological sample and that indicates the presence, absence, absolute amount or concentration, relative amount or concentration, titer, expression level, ratio of measured levels, or the like, of, for, or corresponding to the biomarker in the biological sample. The exact nature of the “level” depends on the specific designs and components of the particular analytic method employed to detect the biomarker.

In another aspect, the present invention is directed to a prostate cancer diagnostic composition including an agent capable of measuring the level of the biomarker and a prostate cancer diagnostic kit including the prostate cancer diagnostic composition.

In the present invention, the kit may include a composition, solution or apparatus suitable for numerical analysis in addition to the agent capable of measuring the level of the prostate cancer diagnostic biomarker composition. For example, the agent may be an antibody specifically binding to the biomarker.

In yet another aspect, the present invention is directed to a prostate cancer diagnostic method including (a) measuring the level of a biomarker including at least one kynurenine pathway metabolite selected from kynurenine and anthranilate in a biological sample from an individual and (b) comparing the measured biomarker level with that in a biological sample from a control.

In the present invention, the biomarker may further include one or more metabolites selected from the group consisting of tryptophan, indoxyl, indole-3-ethanol, indolelactate, isophenoxazine, glutaryl-CoA, (S)-3-hydroxybutanoyl-CoA, acetoacetyl-CoA, and acetyl-CoA.

In the present invention, the biomarker may further include prostate-specific antigen (PSA).

In the present invention, when the level of kynurenine or anthranilate in the sample from the individual is elevated compared to that in the sample from the control, the individual is diagnosed with prostate cancer.

In the present invention, when the level of one or more metabolites selected from the group consisting of tryptophan, indoxyl, isophenoxazine, glutaryl-CoA, (S)-3-hydroxybutanoyl-CoA, acetoacetyl-CoA, and acetyl-CoA in the sample from the individual is elevated compared to that in the sample from the control, the individual is diagnosed with prostate cancer.

In the present invention, when the level of indolelactate or indole-3-ethanol in the sample from the individual is reduced compared to that in the sample from the control, the individual is diagnosed with prostate cancer.

In the present invention, when the level of PSA in the sample from the individual is elevated compared to that in the sample from the control, the individual is diagnosed with prostate cancer.

### EXAMPLES

The present invention will be explained in more detail with reference to the following examples. It will be appreciated by those skilled in the art that these examples are merely illustrative and the scope of the present invention is not construed as being limited to the examples. Thus, the true scope of the present invention should be defined by the appended claims and their equivalents.

### Example 1: Methods

1-1: Sample Collection

The present invention was approved by the Korea University Institutional Review Board (IRB) and was performed in accordance with the ethical guidelines outlined in the Korea University IRB (KU-IRB-15-19-A-1). Informed consent was obtained from all participants whose health data was deposited in the Korean Cancer Prevention Study-II (KCPS-II) Biobank. A pool of 156,701 participants voluntarily underwent private health examinations in one of the 18 centers located in Seoul and Gyeonggi Province in South Korea from 2004 to 2013. Approximately 1300 participants aged 30-60 years were randomly selected, and participants for whom data for essential or metabolic syndrome-related variables (such as fasting glucose levels, body mass index [BMI], and cholesterol levels) was not available were excluded from the study. The present inventors obtained from the health examination record serum total PSA concentration, which was measured immunochemically using the ADVIA Centaur XP Immunoassay (Siemens Diagnostics, Deerfield, Ill.), which is standardized to the World Health Organization international reference standard for PSA (90:10) 96/670 and has an assay range of 0.01-100 ng/mL. Incident data on prostate cancer was ascertained from national cancer registry. A total of 146 qualified subjects were enrolled in the study, with 96 and 50 subjects in the healthy control and PCa groups, respectively. PCa patients were further divided into two groups, based on their prostate-specific antigen (PSA) level (above or less than 4). Details such as age, BMI, and fasting blood sugar, total cholesterol, and PSA levels are shown in Table 1. Fasting blood sugar and total cholesterol levels were measured using the COBAS INTEGRA 800 and 7600 Analyzer (Hitachi, Tokyo, Japan). PCa cases were determined according to the International Classification of Diseases, 10th edition (ICD-10, coded as C61).

TABLE 1

| parameters | control | PCa (PSA < 4) | PCa (PSA > 4) |
|---|---|---|---|
| n | 96 | 25 | 25 |
| age | 63.38 ± 8.29 | 62.52 ± 8.31 | 64.68 ± 8.24 |
| BMI (kg/m²) | 24.20 ± 2.83 | 24.28 ± 3.05 | 24.58 ± 2.9 |
| fasting blood sugar | 101.23 ± 27.71 | 93.92 ± 8.43 | 105.4 ± 40.88 |
| (mg/dL) | | | |
| total cholesterol | 191.86 ± 32.28 | 190.36 ± 33.19 | 189.4 ± 37.84 |
| (mg/dL) | | | |
| PSA level (ng/mL) | 1.13 ± 0.74 | 2.31 ± 0.75* | 14.38 ± 28.01* |

PCa represents prostate cancer patients. PSA > and/or < 4 represents prostate cancer patients with PSA level higher or lower than 4 ng/mL. Values are expressed as mean ± SD.
*Significantly different with control (p < 0.05)

1-2: Sample Preparation and LC-MS Conditions

Serum samples (50 μL) from the healthy control and PCa groups were treated with acetonitrile (1:2, v/v), and centrifuged at 14,000×g for 5 min at 4° C. to separate the proteins. Metabolites were separated using the Agilent 1200 high performance liquid chromatography (HPLC) system (Agilent Technologies, Inc., Santa Clara, Calif., USA) with a Higgins Analytical Targa HPLC C18 100 mm×2.1 mm column, 5 μm particle size (Higgins Analytical, Inc., Mountain View, Calif., USA). The mobile phase A was 0.1% formic acid in water (HPLC grade, Tedia, Ohio, USA) and mobile phase B contained 0.1% formic acid in acetonitrile (HPLC grade). The HPLC gradient was programmed as follows: 0-7 min, 5% B; 7-15 min, gradient was decreased to 2% B; 15-20 min, held at 40% B; 20-24 min, 95% B; and 24-25 min, gradient was decreased to 2% B. The injection volume, flow rate, and column temperature were 5 μL, 0.4 mL/min, and 40° C., respectively. An Agilent 6530 Accurate Mass Q-TOF-LC-MS (Agilent Technologies, Inc.) was used to detect the mass of the metabolites. This system was used to detect ions with mass-to-charge ratio (m/z) of 50-1000 at a resolution of 20,000 over 30 min. The LC was operated with data extraction enabled using aμLCMS software (version 5.9.6, http://clinicalmetabolomics.org/welcome/default/software), which provided a minimum of 3000 reproducible metabolite features, a number of which displayed sufficient mass accuracy to predict the elemental composition. Each chromatogram was defined on the basis of the ion intensity, m/z, and retention time.

1-3: Metabolic Profiling with Univariate and Multivariate Statistical Analysis

The aμLCMS was used to analyze all the metabolite features of the samples for subsequent statistical analyses and bioinformatics. Four analysis groups were generated based the PSA level of PCa patients, as follows: control subjects versus PCa patients with PSA level less than 4 ng/mL (PSA <4) and PCa patients with PSA level higher than 4 ng/mL (PSA >4), control subjects versus PSA <4, control subjects versus PSA >4, and PSA <4 versus PSA >4. Metabolite features from triplicate LC-MS analyses were averaged, log₂ transformed, and normalized using z-transformation. The univariate analysis and false discovery rates (FDR) were calculated to reduce the incidence of false-positives, and Manhattan plots were constructed using MetaboAnalyst 3.0 to identify metabolites, whose levels were significantly different between control vs PSA <4 or control vs PSA >4 or PSA <4 vs PSA >4. For 3-group analyses of control vs PSA <4 vs PSA >4, ANOVA was performed to identify significant metabolites using MetaboAnalyst 3.0. Unsupervised principal component analysis (PCA) was first performed to detect a significant separation shift between all comparison groups. For supervised multivariate analysis, partial least-squares discriminant analysis (PLS-DA) was performed to achieve maximum separation among the groups. The results of PCA and PLS-DA were analyzed by inserting raw matrix data into SIMCA 14.1 (Umetrics AB, Umei, Sweden) and using unit variance (UV) scaling to increase the accuracy of metabolite identification. Thereafter, hierarchical cluster analysis (HCA) was used to separate the metabolic profiles of the comparison groups by inserting raw date into MetaboAnalyst 3.0.

1-4: Metabolic Pathway Analyses

To interpret the data, the metabolites identified as significantly different (with FDR-adjusted P value<0.05) between comparison groups in Manhattan plots (control vs PSA <4 or control vs PSA >4) and ANOVA, were considered important in the identification of potential biomarkers and data regarding these metabolites were subsequently fed into several software platforms. Information for the metabolomes was obtained by Metlin Mass Spectrometry Database (METLIN) (https://metlin.scripps.edu) and the recorded KEGG (Kyoto Encyclopedia of Genes and Genome database; http://www.kegg.jp) numbers served as input for the human metabolomics pathway.

Potentially altered metabolic pathways in control versus PSA <4 versus PSA >4 were identified in KEGG after ANOVA. Significant m/z values obtained from the Manhattan plot in two-group analyses were annotated by Mummichog 2.0.4 to create a potential metabolic network model.

1-5: Targeted Metabolite Profiling

For the identification and quantification of metabolites, the reference standards were purchased from Sigma Chemical Co. (St. Louis, Mo., USA). The standards were weighed accurately, dissolved in methanol/water, as per instructions for the materials, and stored at 4° C. All serum samples of control and PCa patient samples were treated with acetonitrile (1:2, v/v), and centrifuged to precipitate proteins. Tandem mass spectrometry (MS/MS) data were acquired in the positive mode using an Agilent 6550 Accurate Mass UHPLC-Q-TOF-LC-MS (Agilent Technologies, Inc.) with an accompanying ESI interface. The standards and serum samples were first scanned in the mass range (m/z) 50-1000. Collision energy of 0, 5, 10, 15, and 20 V was then used to produce the highly abundant fragment ions of the putative metabolites during product-ion analysis in the positive mode. Chromatography was performed on a C18 100 mm×2.1 mm column (Higgins Analytical, Inc., Mountain View, Calif., USA), at a flow rate of 0.4 μL/min. Concentrations of identified metabolites in serum samples from control or PCa patients were quantified by making the calibration curve of each standard compound with at least eight appropriate concentrations levels. The limit of detection (LOD) and limit of quantification (LOQ) under the present chromatographic conditions were determined at a signal-to-noise (S/N) ratio of 3 and 10, respectively. The analyses were performed in triplicate, and data was presented as mean±SEM. The concentrations of targeted metabolites were calculated by reference to the peak areas of the external standards within the range of LOD and LOQ.

1-6: Statistical Analysis Using GraphPad

Potential metabolites were analyzed using the GraphPad Prism v 5.03 software (La Jolla, Calif.) for measurement of their relative intensities among comparison groups. Data are presented as means±SD, and differences with p values<0.05 were considered statistically significant.

### Example 2; Results

2-1: Subjects' Characteristics

Metabolomics analysis was performed on a total of 50 PCa patients and 96 healthy control subjects. All the control subjects had a PSA level less than 4 ng/mL, while PCa patients were categorized into PSA >4 and PSA <4 groups. On the basis of Student's t test, no statistical differences were observed in the age, BMI (kg/m²), fasting blood sugar (mg/dL), and total cholesterol (mg/dL) among the 3 groups; however, PSA levels were significantly higher in the PCa groups compared to the control subjects, as shown in Table 1.

2-2: Differential Metabolic Phenotype of PCa Patients

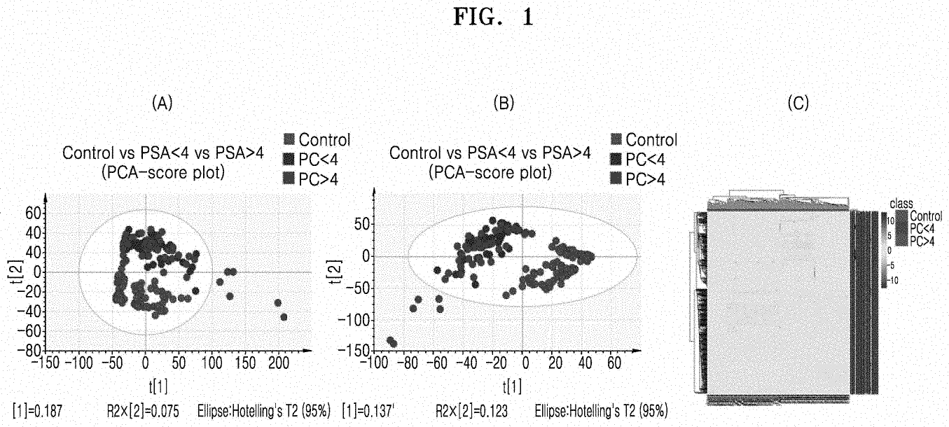

To determine the discriminatory metabolic phenotype between control and PCa patients, the healthy subjects were compared with both PCa groups using unsupervised multivariate principal component analysis (PCA) to detect a significant separation shift between groups. The apLCMS feature table containing 8,855 metabolite features was inserted into SIMCA 14.1 (Umetrics AB, Umei, Sweden) and unit variance (UV) scaling was performed to increase the accuracy of metabolite identification in this data set. Thereafter, supervised multivariate analysis: partial least-squares discriminant analysis (PLS-DA) was performed to achieve maximum separation among the groups. As shown in FIG. 1A and FIG. 1B, the score plot of both the PCA and PLS-DA significantly separated healthy subjects from the two groups of PCa patients. This indicates that the serum metabolome of PCa patients was significantly different from healthy subjects regardless of the PSA level. Additionally, ANOVA test among 3 groups was performed in MetaboAnalyst 3.0. Out of 8,855 features, 1,959 metabolite features were found significant (p<0.05) among the three groups after FDR q=0.05 correction. Furthermore, to better define the differential metabolic profiles and variations among healthy subjects and PCa groups, hierarchical cluster analysis (HCA) using significant metabolite features obtained from ANOVA was performed in MetaboAnalyst 3.0 As shown in FIG. 1C, the metabolite features under red panels at the top, which represent healthy subjects, were clearly separated from the PCa patients (green: PSA <4 and blue: PSA >4).

2-3: Impact of PSA on Metabolic Alteration

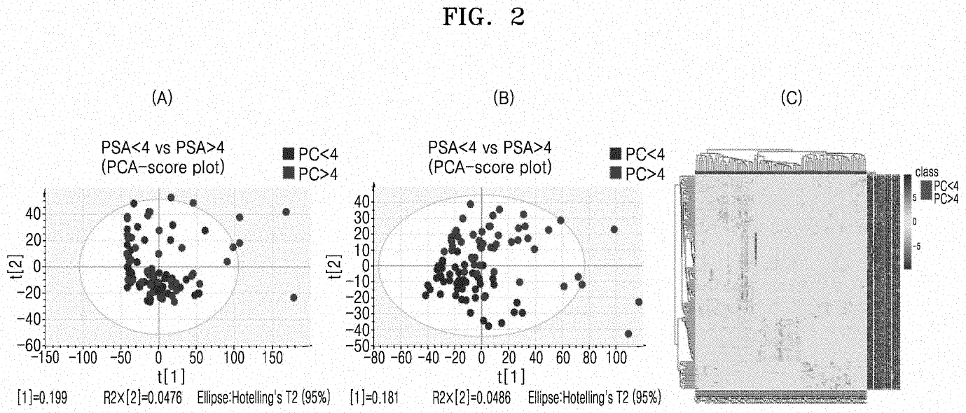

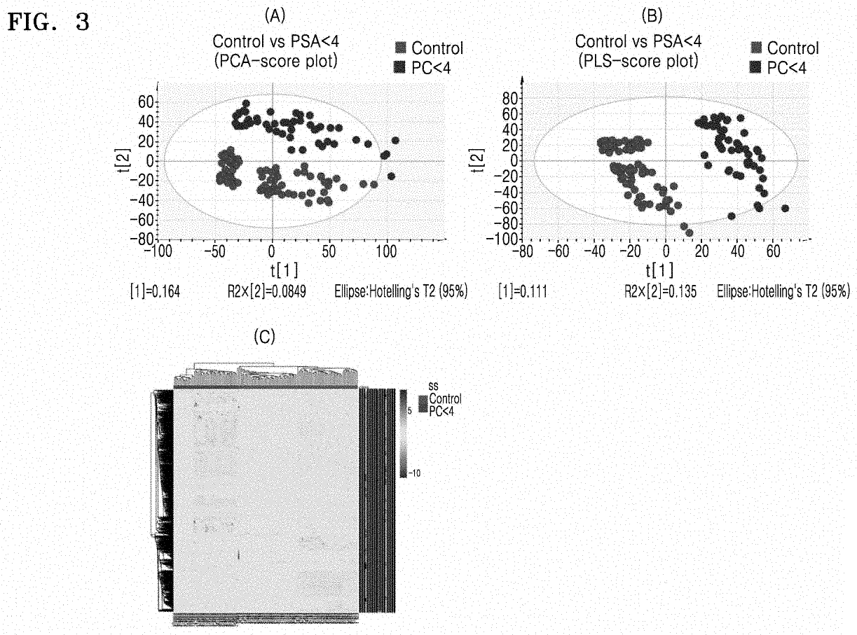

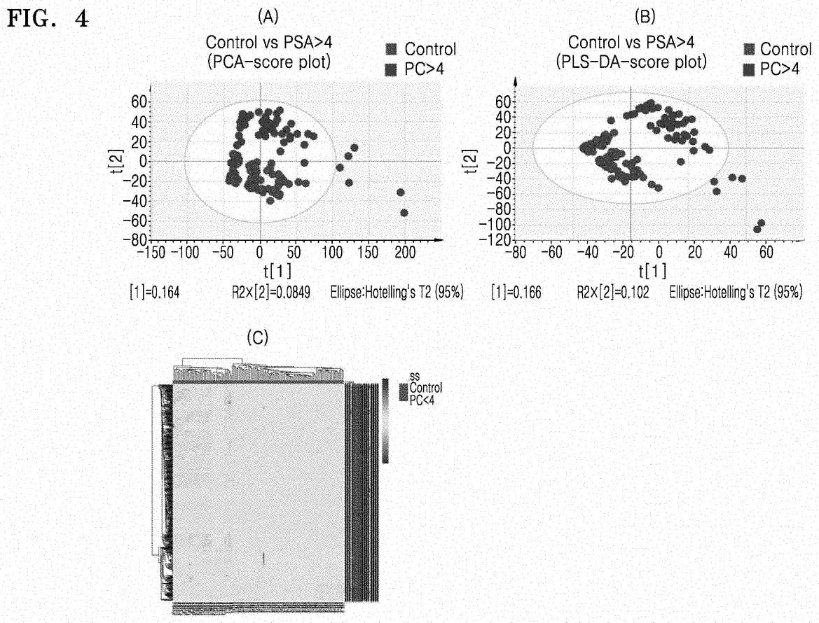

Testing for PSA level in tandem with digital rectal examination (DRE) in elderly men has been the standard method for the detection of PCa. However, despite the PCa patients being aged above 60, the PSA level was high in one-half of the patients, while lower in the other half, suggesting that PSA related cancer-specific sensitivity and specificity exists. Due to variation in the PSA levels of PCa patients, the present inventors focused on determining the role of PSA on metabolic alteration. Although PCA, PLS-DA, and HCA clearly differentiated healthy subjects from PCa patients, none of these parameters were able to separate the PCa PSA <4 and PSA >4 groups. The green panel, representing PCa patients with PSA <4, and the blue panel, representing PCa patients with PSA >4, were not separated by either PCA, PLS-DA, or HCA (FIG. 2A, FIG. 2B and FIG. 2C). This indicates that PSA has a weak impact on metabolic variations. Moreover, the present inventors sought to determine if high PSA levels are specifically responsible for metabolic alterations. Healthy subjects were individually compared with PCa PSA <4 and PSA >4 groups. However, as shown in FIG. 3A, FIG. 3B and FIG. 3C, the PSA <4 group was satisfactorily separated from the healthy controls by PCA, PLS-DA, and HCA, which was similar to that of the result obtained with healthy subjects versus PSA >4 group (FIG. 4A, FIG. 4B and FIG. 4C). This indicates that the differential metabolic profile of PCa patients in comparison with that of the healthy subjects was independent of the PSA level, as both low and high PSA groups of PCa showed similar metabolic profiles. These results were further confirmed when the serum samples of PCa PSA <4 patients were compared with those of PCa PSA >4 patients. As shown in FIG. 2A, PCA was unable to separate the two groups; however, PLS-DA (FIG. 2B) slightly distributed the two groups into two clusters, though the separation distance was not as efficient as in FIGS. 3 and 4. Similarly, the two groups could not be clustered into two groups by HCA (FIG. 2C). It indicates that elevated or low levels of PSA may not strongly affect metabolism.

2-4: Tryptophan Metabolism Metabolites as Specific Signature of High PSA in PCa Patients

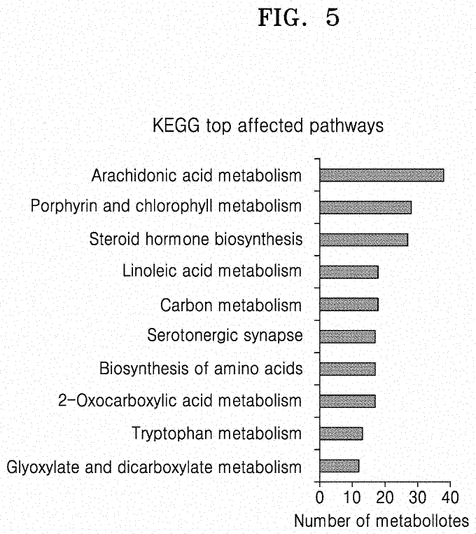

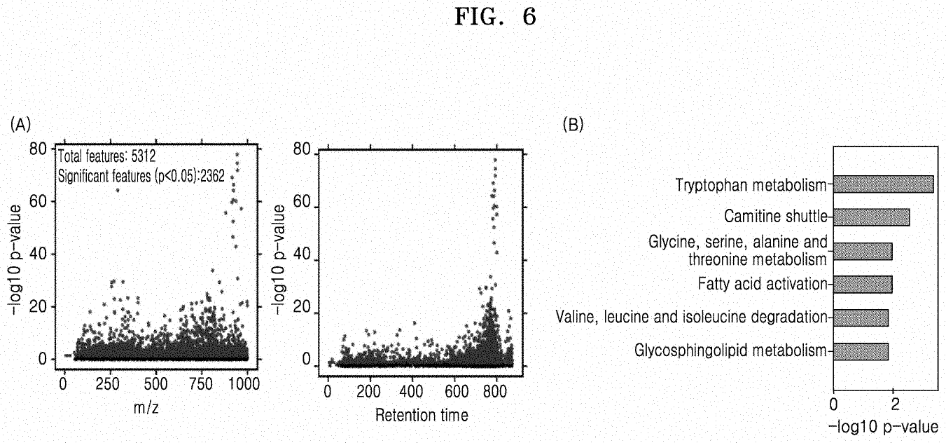

Mummichog, in combination with the results from the analysis of METLIN and KEGG databases, was used to annotate the significant metabolite features obtained from ANOVA and Student's t test. ANOVA yielded 1,959 significant metabolite features among the PSA >4, PSA <4, and control groups. The annotation of these metabolite features in METLIN and pathway analysis in KEGG identified several affected pathways, as shown in FIG. 5. The pathways specifically affected by PSA levels higher than 4 ng/mL were extracted. Metabolite features, whose levels were significantly different between PSA >4 and control, were identified by FDR q=0.05 correction after Student's t test using MetaboAnalyst 3.0. Out of 8,855 features, 1,959 metabolite features were found significant (with FDR-adjusted p<0.05). Furthermore, the significant metabolite features were annotated in the xMSannotator. The KEGG numbers were used for pathway analysis. The top 10 affected pathways among three groups together with the number of hits on the pathways of the metabolites are shown in FIG. 5. PSA >4 and PSA <4 groups were separately analyzed in comparison with the control group using Mummichog. 2,362 significant features were observed in the PSA >4 group, as shown in FIG. 6A. As shown in FIG. 6B, pathway analysis using these significant features showed that tryptophan metabolism was detected with highest impact on the pathway and −log (p) values (0.0005). This indicates relatively high importance of tryptophan metabolites in the specific pathway. A high −log (p) value shows the significance of the pathway based on the statistical experiments. The high impact and −log (p) values indicate pathways with important molecules, whose levels are significantly different between the groups. The −log (p) value for the tryptophan metabolism pathway was high (0.04) in the PSA <4 group compared to the control group. In addition to Mummichog, the tryptophan metabolism pathway was also one of the top 10 KEGG pathways (FIG. 5).

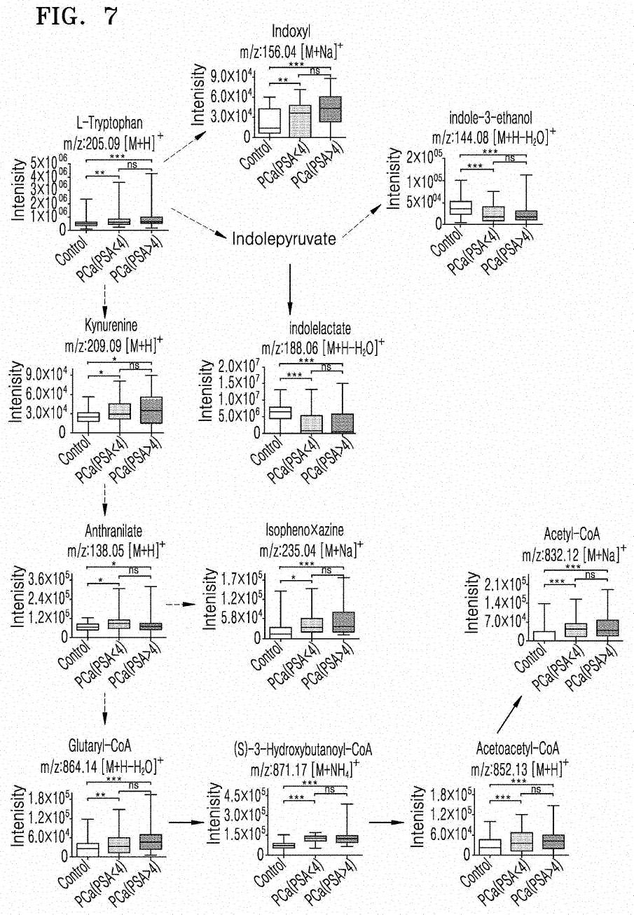

Considering the possible impact of other pathways on PCa, which were identified along with tryptophan metabolism in KEGG or Mummichog analysis, the raw peak intensities of the pathways listed in FIGS. 5 and 6 were measured. Raw intensity was measured by building bar graphs of each metabolite. The metabolites with low intensity in PCa patients were not considered for biomarker validation. Among all the pathway's metabolites, the expression of the following tryptophan metabolism's metabolites along the kynurenine pathway, namely, tryptophan, indoxyl, kynurenine, anthranilate, isophenoxazine, glutaryl-CoA, (S)-3-hydroxybutanoyl-CoA, acetoacetyl-CoA, and acetyl-CoA, was upregulated in correlation with the PSA level in PCa patients (FIG. 7). In contrast, the metabolites of tryptophan metabolism, namely, indolelactate and indole-3-ethanol, through the alternative pathway, were detected at a low intensity in PCa patients compared to the control group (FIG. 4). This further confirmed tryptophan metabolism as the top affected pathway in the PSA >4 compared to the control, as levels of tryptophan metabolites were significantly elevated in the serum of PSA >4 patients. The intensity of these metabolites was slightly higher in the PSA >4 group compared to that in the PSA <4 PCa group, but the p values did not indicate a significant difference (p>0.05). This indicates that the levels of PSA may not strongly alter tryptophan metabolism to differentiate between PCa patients, based on their PSA level.

2-5: Validation of Tryptophan Metabolism's Metabolites in the Serum

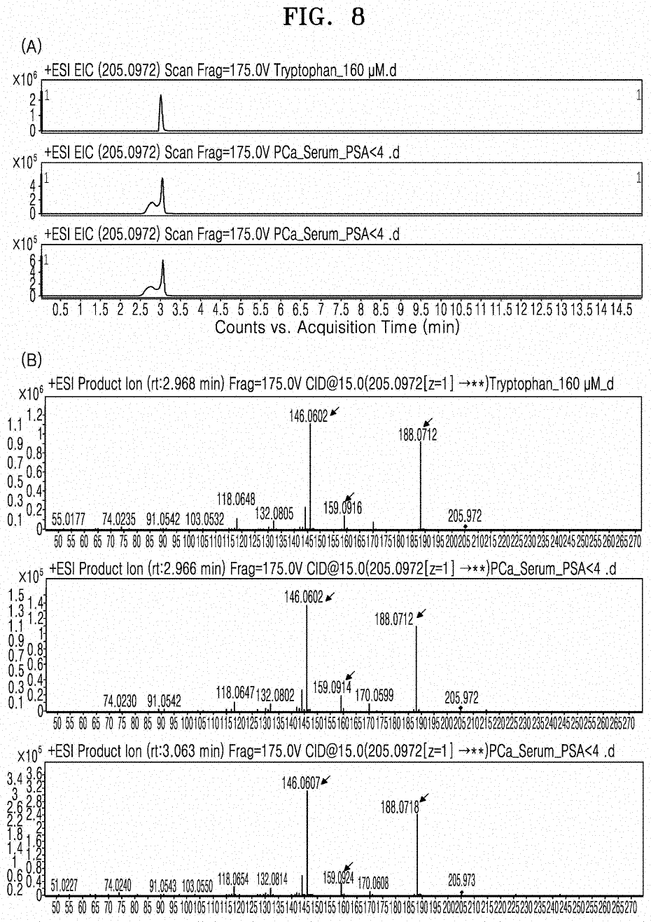

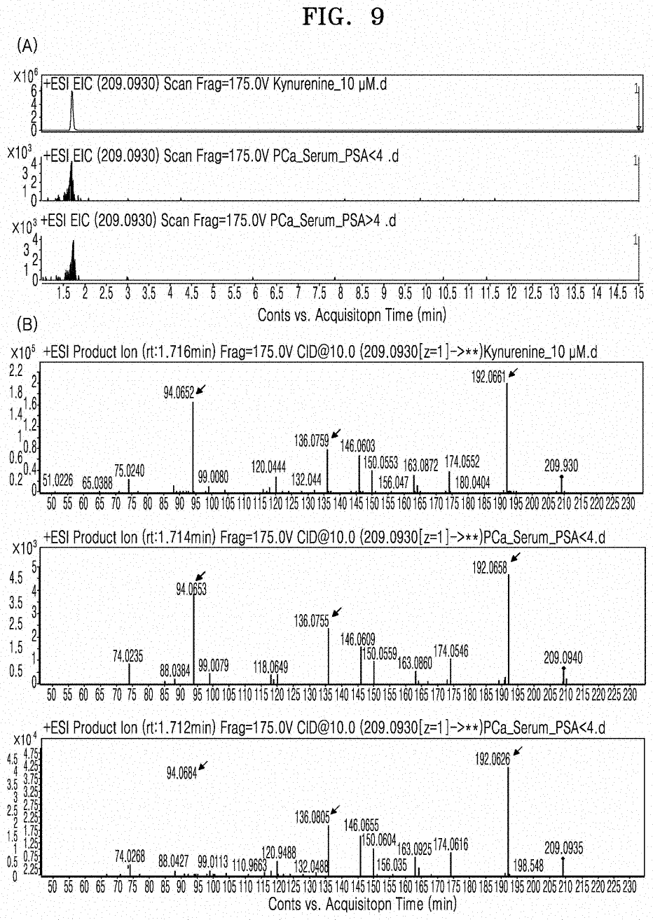

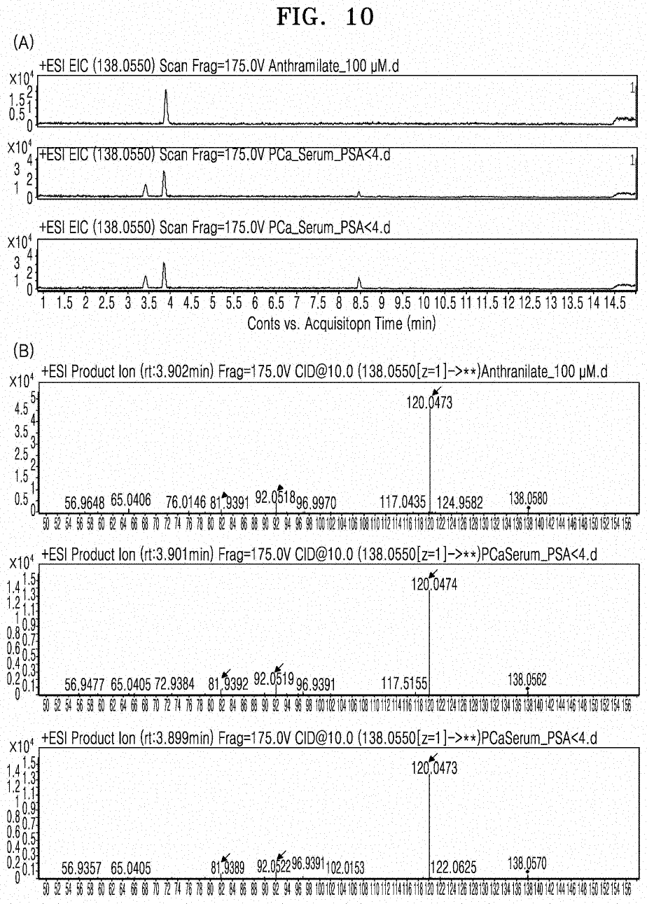

A subset of 11 metabolites was tested by MS/MS. The presence of three key metabolites, namely, tryptophan, kynurenine, and anthranilate, was confirmed in PCa serum samples by comparing the spectra of these metabolites with the standards available in HMDB databases (www.hmdb.ca), as well as with the MS/MS spectra of the standard chemicals. The compounds were scanned followed by product-ion analysis using the collision energy values 0, 5, 10, 15, and 20 eV. The MS/MS spectra of the [M+H]⁺ ion of tryptophan in scan mode is shown in FIG. 8A. MS/MS product-ion analysis of tryptophan in PCa serum samples produced fragment ions at m/z 205.97→m/z 146.06, m/z 188.07, and m/z 159.09, as shown in FIG. 8B. The MS/MS spectra of the [M+H]⁺ ion of kynurenine in scan mode is shown in FIG. 9A. MS/MS product-ion analysis of kynurenine in PCa serum samples produced fragment ions at m/z 209.09→m/z 192.06, m/z 94.06, and m/z 136.07, as shown in FIG. 9B. The MS/MS spectra of the [M+H]⁺ ion of anthranilate in scan mode is shown in FIG. 10A. MS/MS product-ion analysis of anthranilate in PCa serum samples produced fragment ions at m/z 138.05→m/z 120.04, m/z 92.05, and m/z 81.93, as shown in FIG. 10B. Moreover, these metabolites were related to tryptophan metabolism along the kynurenine pathway, which further provides evidence that the kynurenine pathway is strongly affected in PCa patients.

2-6: Determination of Tryptophan, Kynurenine, and Anthranilate in Serum Samples

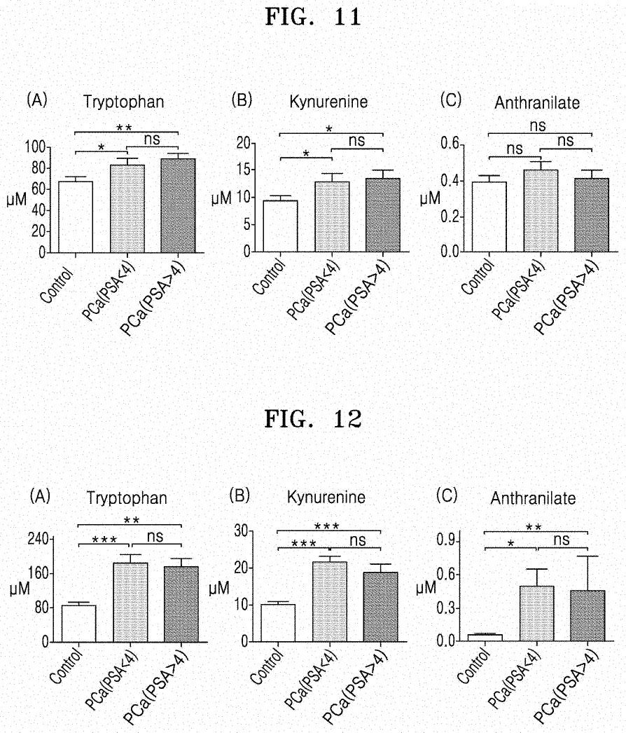

The concentrations of tryptophan, kynurenine, and anthranilate were determined in control and PCa sera, and the results are given in FIG. 11. Their concentrations in serum were calculated by referring to the external standard's calibration curve. In accordance with the LC-MS results (FIG. 7), tryptophan and kynurenine concentrations were found significantly elevated in PCa sera with PSA level <4 or >4 (FIG. 11A and FIG. 11B), while no significant difference was observed among PSA level <4 and >4 sera. Anthranilate showed an upregulated pattern among PCa patients with PSA level <4 or >4; however, the mean values were not significantly different compared to control sera due to high variation among samples (FIG. 11C). This result further confirms elevated kynurenine pathway's metabolites in PCa.

2-7: Validation of Tryptophan, Kynurenine, and Anthranilate in Serum Samples of Training Set

To ensure the consistency of increased tryptophan, kynurenine and anthranilate in PCa patient's sera, the results were further validated in an independent population assigned as training set. The training set was consisting of healthy control (n=100), PCa patients with PSA level <4 (n=37) and PCa patients with PSA level >4 (n=13).

In training set sera the quantified concentration of tryptophan, kynurenine, and anthranilate were determined. Interestingly, in accordance with the results obtained in FIG. 11, the training set showed the exact same upregulation of tryptophan, kynurenine, and anthranilate in PCa sera with PSA level <4 or >4 (FIG. 12A, FIG. 12B and FIG. 12C), while no difference was observed in in PCa sera with PSA level <4 and >4. More interestingly, anthranilate which was previously (FIG. 12C), observed with no significant difference in compared groups, showed significant elevation in PCa sera of the test set (FIG. 12C).

Although the particulars of the present disclosure have been described in detail, it will be obvious to those skilled in the art that such particulars are merely preferred embodiments and are not intended to limit the scope of the present invention. Therefore, the true scope of the present invention is defined by the appended claims and their equivalents.

What is claimed is:

1. A prostate cancer diagnostic biomarker composition comprising at least one kynurenine pathway metabolite as an active ingredient wherein the kynurenine pathway metabolite is kynurenine and/or anthranilate.

2. The prostate cancer diagnostic biomarker composition according to claim 1, further comprising one or more metabolites selected from the group consisting of tryptophan, indoxyl, indole-3-ethanol, indolelactate, isophenoxazine, glutaryl-CoA, (S)-3-hydroxybutanoyl-CoA, acetoacetyl-CoA, and acetyl-CoA.

3. The prostate cancer diagnostic biomarker composition according to claim 1, further comprising prostate-specific antigen (PSA).

4. A prostate cancer diagnostic composition comprising an agent capable of measuring the level of the biomarker composition according to claim 1.

5. A prostate cancer diagnostic kit comprising the prostate cancer diagnostic composition according to claim 4.

6. A method for providing information on prostate cancer diagnosis, comprising: (a) measuring the level of a biomarker comprising at least one kynurenine pathway metabolite selected from kynurenine and anthranilate in a biological sample from an individual; and (b) comparing the measured biomarker level with that in a biological sample from a control.

7. The method according to claim 6, wherein the biomarker further comprises one or more metabolites selected from the group consisting of tryptophan, indoxyl, indole-3-ethanol, indolelactate, isophenoxazine, glutaryl-CoA, (S)-3-hydroxybutanoyl-CoA, acetoacetyl-CoA, and acetyl-CoA.

8. The method according to claim 6, wherein the biomarker further comprises prostate-specific antigen (PSA).

9. The method according to claim 6, wherein when the level of kynurenine or anthranilate in the sample from the individual is elevated compared to that in the sample from the control, the individual is diagnosed with prostate cancer.

10. The method according to claim 7, wherein when the level of one or more metabolites selected from the group consisting of tryptophan, indoxyl, isophenoxazine, glutaryl-CoA, (S)-3-hydroxybutanoyl-CoA, acetoacetyl-CoA, and acetyl-CoA in the sample from the individual is elevated compared to that in the sample from the control, the individual is diagnosed with prostate cancer.

11. The method according to claim 7, wherein when the level of indolelactate or indole-3-ethanol in the sample from the individual is reduced compared to that in the sample from the control, the individual is diagnosed with prostate cancer.

12. The method according to claim 8, wherein when the level of PSA in the sample from the individual is elevated compared to that in the sample from the control, the individual is diagnosed with prostate cancer.
